(19) United States
(12) Patent Application Publication
(10) Pub. No.: US 2014/0148512 A1
(43) Pub. Date: May 29, 2014

(54) Soap capable of treating seborrheic keratoses, keratosis pilaris, keratoderma, and related conditions and methods of making and using

(71) Applicant: BURKE PHARMACEUTICALS, INC., Hot Springs, AR (US)

(72) Inventors: Dowling B. Stough, Hot Springs, AR (US); Timothy E. Dugan, Bryant, AR (US); Michael Scott Krause, Essex Junction, VT (US); Shanna Marie Cullinane, South Burlington, VT (US)

(73) Assignee: Burke Pharmaceuticals, Inc., Hot Springs, AR (US)

(21) Appl. No.: 13/694,403

(22) Filed: Nov. 29, 2012

(51) Int. Cl. A61K 31/20

(52) U.S. Cl. CPC A61K 31/20
USPC 514/558

(57) ABSTRACT

Embodiments include a soap capable of treating seborrheic keratoses, keratosis pilaris, keratoderma, and/or related conditions, comprising a soap base and additives including pumice and polyethylene. Embodiments further include a method of making and using the soap, comprising providing the soap base and the additives including pumice and polyethylene, mixing the soap base and the additives together, and forming the soap from the soap base and the additives. Embodiments also include a method of using the soap, comprising providing the soap comprising the soap base and additives including pumice and polyethylene, and placing the soap in rubbing contact with the seborrheic keratoses, keratosis pilaris, keratoderma, or related conditions.

### BACKGROUND OF THE INVENTION

1. Field of the Invention

Embodiments generally relate to soap capable of treating seborrheic keratoses (also seborrheic keratosis), keratosis pilaris, keratoderma, and related conditions.

2. Description of the Related Art

Seborrheic keratosis, also known as seborrheic verruca and senile wart, is a type of benign skin growth that begins in keratinocytes. Seborrheic keratoses, which often start out as small, rough areas, may eventually develop a thick, wart-like surface. Although the growths are not harmful, they can be unattractive and may cause embarrassment. The growth or lesion may itch, and there is risk of localized infection caused by picking at the lesion. Some reasons that seborrheic keratoses may be removed are because they itch, they interfere with or are irritated by clothing or jewelry, they cause emotional or physical discomfort, or they are cosmetically unacceptable.

Limited treatment options are available for seborrheic keratoses, all of which are either ineffective or invasive procedures that must be performed by a physician. Some small lesions can be treated with light electrocautery, while larger lesions can be treated with electrodesiccation and curettage (electrosugery/cutterage), shave excision, or cryotherapy or cryosurgery.

With electrodesiccation and curettage, a round dull instrument (curette) of varying sizes is used to scrape off the cancer down to the dermis. The scraping is then paused while an electrosurgical device like a hyfrecator is used next. Electrocoagulation (electrodesiccation) is then performed over the raw surgical ulcer to denature the protein of the dermis. The curette is used again over the surgical ulcer to remove the denatured dermis down to living tissue. The electrodesiccation is performed again, and the cycle is repeated until the surgeon is comfortable of the depth of the destruction and that adequate surgical margin has been obtained.

Electrosurgery uses an electrical current to scrape the growth off. Electrocauterization or electrocautery is the process of destroying tissue using heat conduction from a metal probe heated by electric current.

Another way to remove seborrheic keratoses is by shave excision, or shaving the growths off, such as by using a flexible razor blade going just deep enough to get only the seborrheic keratosis cells and leave normal skin. Scarring is possible with shave excision when too much normal skin is shaved off. After the lesion is shaved, a chemical agent such as aluminum chloride or silver nitrate is applied to the wound to stop any bleeding. Silver nitrate is a dark brown color, and consequently, the resulting wound after the shave is dark brown. Although this color will usually go away after the skin repairs, some of that pigment can remain, leaving undesirable coloring on the skin.

With cryosurgery, the seborrheic keratosis growth is frozen off using liquid nitrogen. Liquid nitrogen freezes and destroys the cells but leaves the connective tissue foundation intact. The lesion frozen forms a blister as the water is released from the now-dead cells then crusts over as that water dries. When the crust falls off after several days, the skin underneath has begun to repair itself. Liquid nitrogen can leave a scar as the repaired skin may have more or less pigment producing cells.

Therefore, current treatment options for seborrheic keratosis are invasive and expensive surgical procedures and include risk of scarring and/or disadvantageous skin coloring. There is a need for a less invasive and less expensive treatment option for seborrheic keratosis which reduces or eliminates risk of scarring and skin coloring.

Keratosis pilaris (“KP”), also known as follicular keratosis or chicken skin, is a common skin condition in which keratin, which is a protein in the skin, forms hard plugs within hair follicles. KP, which may be manifested by the appearance of rough, slightly red, small, hard bumps on the skin, occurs when the human body produces excess keratin. The excess keratin surrounds and entraps the hair follicles in the pore, causing the formation of hard plugs in a process known as hyperkeratinization. Many KP bumps contain an ingrown hair that has coiled, which is a result of the keratinized skin preventing the hair from exiting so that the hair grows encapsulated inside the follicle.

KP is often cosmetically unacceptable, and the person suffering from this condition may want treatment of the condition. Typical treatment options may include moisturizing lotions to soothe the skin and help it look better, skin creams that contain urea, lactic acid, glycolic acid, salicylic acid, tretinoin, or vitamin D, or steroid creams to reduce redness. With these available treatment options, improvement often takes months and the bumps are likely to come back.

What is therefore needed is an effective, accessible, less expensive treatment for seborrheic keratoses.

What is also needed is a treatment for seborrheic keratoses which does not require surgery or physician intervention.

What is also needed is an effective, accessible treatment for keratosis pilaris.

Keratoderma is a hornlike skin condition, or a disease of the horny layer of the skin of the palms and soles. The appearance or keratoderma is usually of a vesico-pustular waxy skin lesion with a yellow brown color. These skin lesions may join together to form larger crusty plaques with desquamating edges. The following treatments for keratoderma may be used in an attempt to soften the thickened skin and make them less noticeable: emollients, eratolytics (e.g. 6% salicylic acid in 70% propylene glycol), topical retinoids, topical vitamin D ointment (calcipotriol), and systemic retinoids (acitretin). These treatments either are not very effective, are not very easily accessible, or require physician intervention.

What is needed is an effective, accessible treatment for keratoderma.

What is also needed is a treatment for keratoderma which does not require physician intervention.

### SUMMARY OF THE INVENTION

Embodiments generally include soap for treating seborrheic keratoses and related conditions.

Embodiments further include soap for treating keratosis pilaris and related conditions.

Embodiments further include soap for treating keratoderma and related conditions.

Embodiments further include a system and method for making soap capable of treating seborrheic keratoses, keratosis pilaris, keratoderma, and/or related conditions.

Embodiments further include a method of use a soap for treating seborrheic keratoses, keratosis pilaris, keratoderma, and/or related conditions.

Some embodiments generally include a soap which smoothes roughly textured skin and skin lesions.

Some embodiments generally include a soap which smoothes rough, thick skin on the feet.

Some embodiments generally include a soap which employs abrasive beads to smooth rough skin.

Embodiments generally include soap for treating seborrheic keratoses, keratosis pilaris, keratoderma, or related conditions, comprising a soap base comprising one or more oils or fatty acids; and additives comprising pumice and polyethylene.

Embodiments also generally include a method of making soap for treating seborrheic keratoses, keratosis pilaris, keratoderma, or related conditions, comprising providing a soap base comprising one or more oils for saponifying the soap; providing additives comprising pumice and polyethylene; mixing the soap base and the additives together; and forming the soap from the soap base and the additives.

Embodiments also generally include a method of using a soap for treating seborrheic keratoses, keratosis pilaris, keratoderma, or related conditions, comprising providing the soap comprising a soap base comprising one or more oils or fatty acids for saponifying the soap, and additives comprising pumice and polyethylene; and placing the soap in rubbing contact with the seborrheic keratoses, keratosis pilaris, keratoderma, or related conditions.

### BRIEF DESCRIPTION OF THE DRAWINGS

So that the manner in which the above-recited features of embodiments can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a table listing ingredients and their weight percents in one example of a soap of embodiments.

FIG. 2 is an embodiment of a system and method for making a soap of embodiments.

FIG. 3 is a table listing ingredients and their weight percents in a second example of a soap of embodiments.

FIG. 4 is a table listing ingredients and their weight percents in a third example of a soap of embodiments.

FIG. 5 is a table listing ingredients and their weight percents in a fourth example of a soap of embodiments.

### DETAILED DESCRIPTION

Embodiments generally include soap, a method of making the soap, and a method of using the soap. The soap may include pumice and polyethylene to allow treatment of seborrheic keratoses and related conditions and/or keratosis pilaris and related conditions and/or keratoderma and related conditions. The pumice and polyethylene may be present in the soap in a ratio of 2:1 in their respective percents by weight in the final soap product (e.g., when the weight percent of pumice in the final soap product is 2 percent, the weight percent of pumice in the final soap product is 1 percent).

The soap may include a soap base B and one or more additives A. The soap base B may also be termed a base soap. The soap base or base soap B may be any soap base or base soap known to those skilled in the art.

Although they both have emulsifiers and surfactants, soaps are very different from detergents. Soaps are made from fat or vegetable oils that have been saponified with sodium or potassium hydroxide. The soap base or base soap B is the sodium or potassium salt of a fatty acid prior to the addition of scent or other nonessential ingredients. The sodium or potassium comes from a strong base such as sodium or potassium hydroxide. The fatty acid may be beef fat(s) or vegetable fat(s), found in palm oil or coconut oil or tallow.

Sodium palmate and sodium cocoate are fatty acids derived from palm and coconut oil which may be included in the soap base. Sodium cocoate is a generic name for the mixture of fatty acid salts resulting from the reaction of coconut oil with sodium hydroxide. Package labels may use the names coconut oil, fatty acids, coco and sodium salts for sodium cocoate. Sodium cocoate may be a surfactant and emulsifying agent.

Sodium palmate results upon the reaction of palm oil with sodium hydroxide, this process called saponification. Saponification occurs when a fatty acid, such as palm oil, reacts with an alkali, such as sodium hydroxide. Glycerol and sodium palmate are the end products of this reaction. Therefore, glycerol is a byproduct of the reaction of palm oil with sodium hydroxide. Sodium palmate may act as a surfactant and emulsifying agent and is a key soap ingredient.

Some example ingredients in the soap base may include fatty acids or oils to saponify the soap as described above, for example sodium palmate (Chemical Abstracts Service (“CAS”) No. 61790-79-2), sodium palmkernelate, sodium tallowate, sodium rapeseedate (CAS No. 68440-17-5), and/or palm oil; and sodium cocoate (CAS No. 61789-31-9) or coconut oil, and/or synthetic soap bases such as sodium cocoyl isethionate.

The soap base B may also include optional water (aqua) (CAS No. 7732-18-5) and optionally one or more preservatives and chelating agents such as tetrasodium ethylenediaminetetraacetic acid (tetrasodium EDTA) (CAS No. 64-02-8) and/or tetrasodium etidronate (CAS No. 3794-83-0). In one embodiment, tetrasodium EDTA and tetrasodium etidronate may both be present in the soap base, each in approximately the same weight percent.

Tetrasodium EDTA may be used to sequester metal ions to allow them to remain in formulas but lose their ability to react with other ingredients, to improve the soap's stability in air as a sequestering agent, to prevent potential defoaming action of hard ions, to reduce soap scum, and/or to improve lather and foaming action of the soap.

Tetrasodium etidronate, also tetrasodium (1-hydroxyethylidene) bisphosphonate, has the following chemical formula:

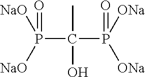

Diosodium etidronate has the following chemical formula:

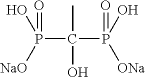

Tetrasodium etidronate and the related disodium etidronate are chelating agents and sequestering agents that attach to metal ions to keep them from reacting with other compounds in a mixture, as well as to prevent them from precipitating out of solution as insoluble scums. Tetrasodium etidronate may also be used as a water softener in soaps to prevent soap scums and bathtub rings by locking up the calcium and magnesium in the water. Following are compounds with similar functions: sodium carbonate, sodium EDTA, sodium citrate, phosphoric acid, and tetrasodium pyrophosphate.

In the soap base B, glycerin (CAS No. 56-81-5) and sodium chloride (CAS No. 7647-14-5) may exist as a byproduct of saponification of two vegetable oils or a byproduct of the reaction.

The soap may include one or more additives A in addition to the soap base B. The additives A may include polyethylene (CAS No. 1332-09-08) to increase exfoliation ability of the soap and pumice (CAS No. 9002-88-4) to provide more even exfoliation feel or drag to the soap than polyethylene would provide. The polyethylene in the soap may be a rough bead cut, or a tumbled or rounded polyethylene bean. In some embodiments, the pumice and polyethylene are included in the soap final product at a ratio of 2:1 in their respective percents by weight in the final soap product.

Another additive A in the soap may be palmitic acid (CAS No. 57-10-3) or coconut acid, which is a performance or processing acid and a fatty acid. Any other similar performance and processing acid known to those skilled in the art may be used in the soap in addition to or in lieu of the palmitic acid or coconut acid.

Palmitic acid is used to produce soap using sodium palmitate, which is obtained by saponification of palm oil. Palm oil, rendered from the coconut palm nut, is treated with sodium hydroxide (in the form of caustic soda or lye), which causes hydrolysis of the ester groups, and produces glycerol and sodium palmitate.

Other additives A such as one or more colorants may be included in the soap to provide a color to the soap. The one or more colorants may include, for example, one or more cosmetic colorant(s) such as titanium dioxide (colour index (CI) 77891) (TiO₂) (CAS No. 13463-67-7) and/or chromium hydroxide green (CI 77289) (CAS No. 12001-99-9, one or more iron oxides (CI 77499) (CAS No. 1317-61-9), and/or Yellow 10 (CI 47005) (CAS No. 8004-92-0). Any types and combinations of colorants which are known to those skilled in the art may be present in the soap as additives A.

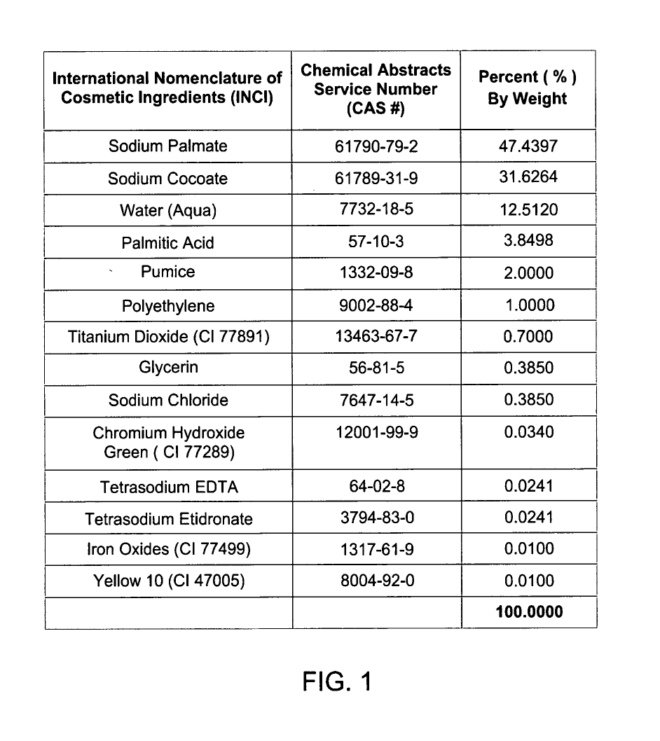

FIG. 1 is a table listing ingredients and their weight percents in one example of a soap product of embodiments. The International Nomenclature of Cosmetic Ingredients (INCI) Nomenclature column lists ingredients of one embodiment of soap. Any equivalent ingredients which perform the same or similar purpose may be used in lieu of the listed ingredients, including those alternative ingredients described herein. The CAS # column lists the Chemical Abstracts Service (CAS) numbers of this embodiment of the soap. The percent by weight is the percent by weight each component is present in this embodiment of the soap product, as a component weight percent of the total weight of the soap product. The weight percents of components listed in FIG. 1 are merely exemplary and may be approximate, and including relative amounts of components' weight percents which are not the same as those listed is within the scope of embodiments. Additionally, the weight percents of components listed in FIG. 1 may fall within a range surrounding the weight percents listed in FIG. 1 where the final mixture of ingredients remains effective.

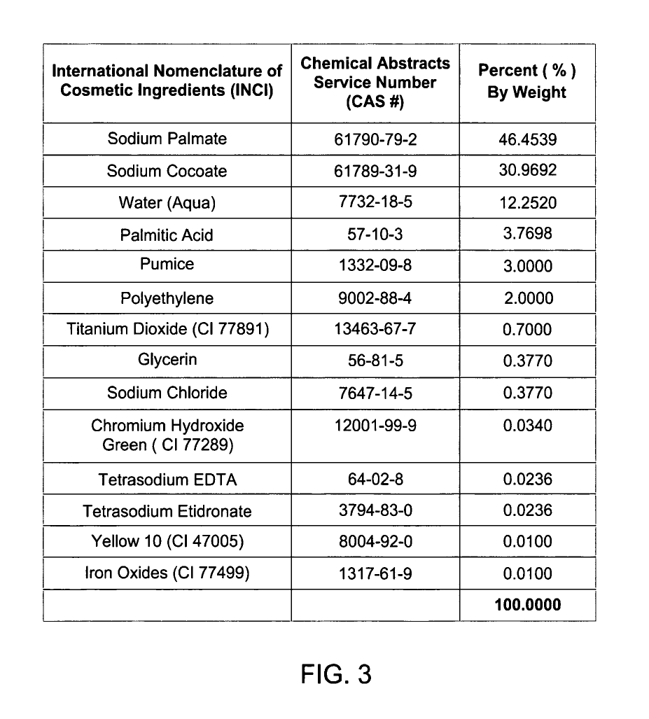

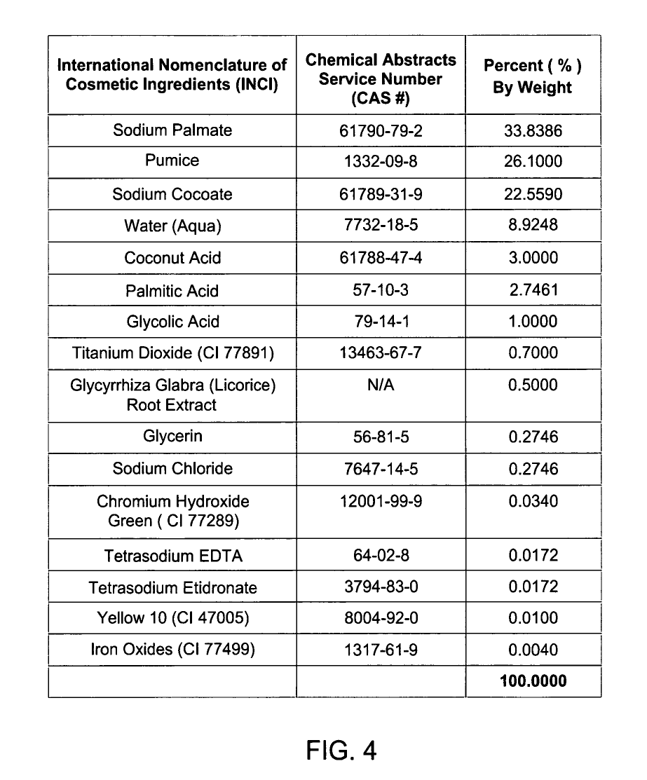

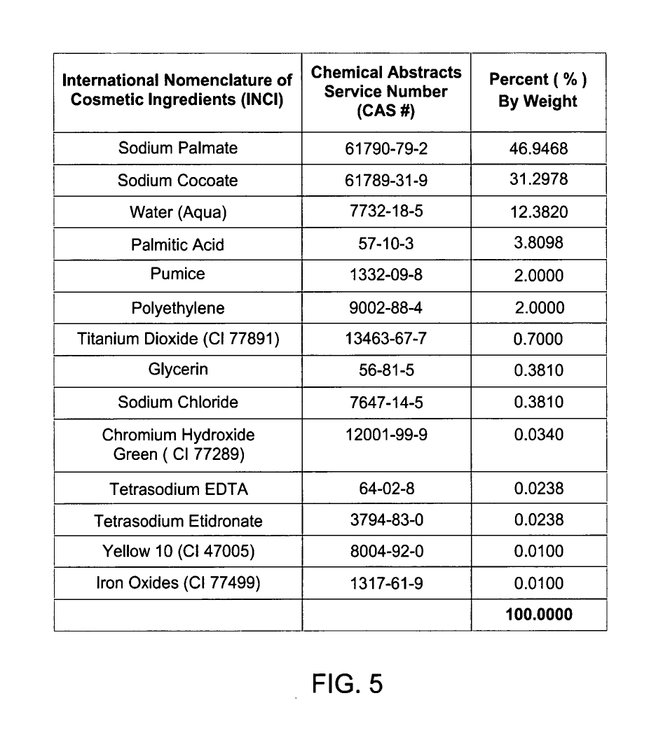

FIG. 3 is a table listing ingredients and their weight percents in a second example of a soap product of embodiments, FIG. 4 is a table listing ingredients and their weight percents in a third example of a soap product of embodiments, and FIG. 5 is a table listing ingredients and their weight percents in a fourth example of a soap product of embodiments. The International Nomenclature of Cosmetic Ingredients (INCI) Nomenclature column lists ingredients of these embodiments of soap. Any equivalent ingredients which perform the same or similar purpose may be used in lieu of the listed ingredients, including those alternative ingredients described herein. The CAS # column lists the Chemical Abstracts Service (CAS) numbers of these embodiments of the soap. The percent by weight is the percent by weight each component is present in each embodiment of the soap product, each figure representing an embodiment of the soap product, as a component weight percent of the total weight of the soap product. The weight percents of components listed in FIGS. 3-5 are merely exemplary and may be approximate, and including relative amounts of components' weight percents which are not the same as those listed is within the scope of embodiments. Additionally, the weight percents of components listed in FIGS. 3-5 may fall within a range surrounding the weight percents listed in FIGS. 3-5 where the final mixture of ingredients remains effective.

FIGS. 3 and 5 show the same ingredients as are shown in FIG. 1, but with some or all of the ingredients having different weight percents in the soap product. In FIG. 3, the weight percent of pumice is at or around three percent while the weight percent of polyethylene is at or around two percent, and in FIG. 5 the weight percent of pumice is at or around two percent while the weight percent of polyethylene is at or around two percent.

FIG. 4 illustrates an example embodiment of a soap product having different ingredients than the soap product of FIGS. 1, 3, and 5. The soap product of FIG. 4 includes pumice but does not include polyethylene. Additionally, the soap base B includes coconut acid (CAS #61788-47-4), glycolic acid (CAS #79-14-1), and glycyrrhiza glabra (licorice) root extract.

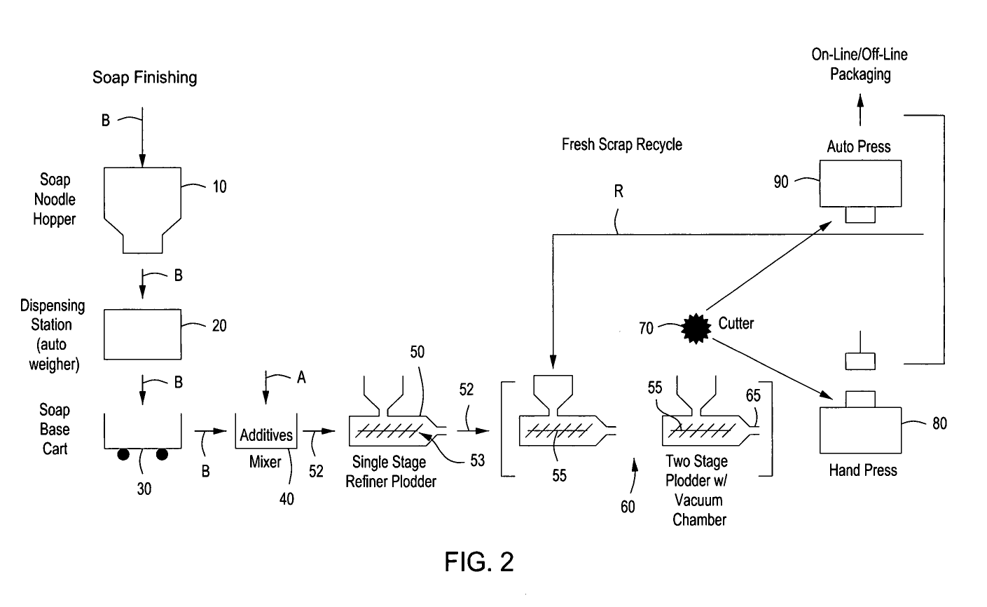

Any system and method for making the soap which is known to those skilled in the art as a system and method for making soap may be utilized to make the soap of embodiments. FIG. 2 is an example embodiment of a system and method for making a soap of embodiments. The system may include a soap noodle hopper 10 or other storage unit for storing and dispensing a soap base B, a dispensing station 20 or dispensing and weighing device for weighing the soap base B and dispensing the desired weight of soap base B into a soap base cart 30, and the soap base cart 30, which may be portable with one or more wheels on its bottom, for storing soap base B prior to its mixing with the one or more additives A. Any or all of these components may be eliminated and functions of the components combined in one unit in other embodiments.

The system may further include a mixer 40, which may be a paddle wheel mixer, for example a paddle wheel mixer including one shaft with multiple paddles on the shaft for moving material within the mixer to mix the material together. In some embodiments, the mixer 40 may be capable of holding 200-kilogram batches at a time.

Disposed downstream of the mixer 40 may be a preliminary plodder 50 or preliminary refining plodder such as a single stage refiner plodder. A two-stage plodder 60 with a vacuum chamber may be disposed downstream of the preliminary plodder 50. The two-stage plodder 60 may include one or more screws 55 within the plodder chamber and one or more compression cones and one or more extrusion plates at an exit end 65 of the two-stage plodder 50. The one or more compression cones and one or more extrusion plates may be used to extrude the exiting material and obtain a continuous flow of soap with the proper cross-section. Any self-finishing plodder known to those skilled in the art may be used for the plodder 60 of the system of embodiments.

The system may include one or more cutters 70 or cutting devices for cutting the soap into billets or into the desired or proper length (e.g., the length of soap to properly fill the die), which may be any device known to those skilled in the art capable of cutting soap into billets or into the desired or proper length (e.g., the length of soap to properly fill the die). One or more pressing molds (dies) such as one or more hand presses 80 and/or one or more auto presses 90 may also be included with the system for stamping the soap into the desired shape.

In a method of making the soap of embodiments, a soap base B containing one or more oils (e.g., vegetable oils) or fatty acids to saponify the soap such as sodium palmate, palm oil, its equivalents, and/or its other alternatives listed herein, and/or sodium cocoate, coconut oil, its equivalents, and/or its other alternatives listed herein; water; one or more preservatives and/or chelating agents such as tetrasodium EDTA, its equivalents, and/or other alternatives listed herein and/or tetrasodium etirdronate, its equivalents, and/or other alternatives listed herein; and the byproducts of saponification, glycerin and sodium chloride is formed by mixing the one or more oils or fatty acids, water, and one or more preservatives and/or chelating agents together. Upon saponification, new ingredients appear due to the chemical reaction than what ingredients were present in the feed. Glycerin is a byproduct of saponification of the two oils (e.g., vegetable oils) such as palm oil (sodium palmate) and coconut oil (sodium cocoate). Sodium chloride is a byproduct of the soap base reaction. Any other soap base B known to those skilled in the art may be utilized with embodiments. The soap base B is a crude homogenization of the initial ingredients.

The soap base B, sometimes termed the “raw soap noodles,” may be added to the soap noodle hopper 10 or other storage and/or dispensing unit. The hopper 10 may gravity feed the soap base B into the dispensing station 20 and weighing device, which may be an auto weighing device. The weighing device may pre-weigh the soap base B and deliver the desired amount of soap base B into the soap base cart 30. The soap base B present in the soap base cart 30 may be delivered into the mixer 40. It is also within the scope of embodiments that the storage, dispensing, weighing, and/or delivering functions may be performed by just one, two or more than three devices rather than requiring the shown three devices of the hopper 10, dispensing station and weighing device 20, and soap base cart 30.

Also added into the mixer 40 are one or more additives A. The additives A may include the pumice and polyethylene which provide the exfoliation properties that make the soap capable of aggressively and sharply attacking the seborrheic keratoses and related conditions and/or keratosis pilaris and related conditions and/or keratoderma and related conditions. In one embodiment, the pumice and polyethylene are present in the soap product in a ratio of 2:1 by weight percent of the total soap product, for example, when the weight percent of pumice in the total soap product is two percent (2%) the weight percent of polyethylene is one percent (1%).

Additionally, the one or more additives A may include one or more performance and processing acids such as palmitic acid, its alternatives described herein, and/or its equivalents, and/or one or more optional colorants. The one or more colorants are optional and not required in embodiments. The one or more colorants may include one or more of the following, for example: titanium dioxide, chromium hydroxide green, iron oxide(s), and/or yellow 10. Any other colorant known to those skilled in the art may be included with the additives to manipulate the color the eventual soap product in addition to or instead of any of the colorants listed herein.

Each of the additives A may be added to the mixer 40 separately or together (or with any combination of the additives mixed together, each combination added separately or together to the mixer 40). In one embodiment, each of the additives A is added into the mixer 40 separately. The amount of each additive A to be added into the mixer is determined and calculated based on the weight percent of components present in the soap base B and the desired weight percents of components in the desired soap product after adding the additives A. The formulation quantities of additives A, including any perfumes and coloring materials, are added to the mixer 40. Although in one embodiment the soap base B is added to the mixer 40 prior to the additives A being added to the mixer 40, it is within the scope of embodiments that the additives A may be added to the mixer 40 prior to the soap base B addition. Additionally, in an alternate embodiment, the ingredients in the soap base B may be added directly to the mixer 40 and optionally mixed therein rather than the soap base B being pre-mixed. In other embodiments, the pre-mixed soap base B is added directly to the mixer 40.

The mass including the soap base B and the additives A is mixed in the mixer 40, e.g. via rotation of the shaft and its paddles in the mixer 40, until the mixture is generally consistent. Once the mixture within the mixer 40 is generally consistent, the soap mixture 52 is gravity fed into the preliminary plodder 50, which may be a single stage refiner plodder with one or more plodder screws 53 therein and one or more mesh screens. The soap mixture 52 is then partially refined in the preliminary plodder 50 and forced by the plodder screw(s) 53 through the one or more mesh screens. Any refining plodder usable for soap-making is within the scope of embodiments for preliminary plodder 50.

The soap 52 is transferred from the preliminary refining plodder 50 to the two-stage plodder 60 with a vacuum chamber. The two-stage plodder 60 may include one or more plodder screws 55 therein for moving the material therein. At or near the exit 65 of the second stage of the two-stage plodder 60, the soap 52 may be extruded through one or more compression cones and one or more extrusion plates to obtain a continuous flow of soap product P with the proper cross-section.

Upon its exit from the two-stage plodder 60, the soap product P is cut into the desired length, e.g., cut into billets of the proper length to fill the pressing mold(s) or die(s). One or more pressing molds or dies such as a hand press 80 and/or auto press 90 are employed to stamp the soap billets into the desired shape and, if desired, incorporate any logos or patterns into the soap bar. The hand press 80 involves pressing the soap into the desired shape by hand, and the auto press 90 involves automated pressing of the soap into the desired shape using an automated, powered system.

Optionally, on-line or off-line packaging may be employed after the one or more pressing molds and/or dies stamp the soap billets into the desired shape. The stamped soaps may be placed into lined plastic trays and located into optional intermediate storage for curing until ready for packing, or instead may be automatically wrapped on line. After curing the stamped soaps as necessary, the stamped soap bars may be packaged if desired.

Scraps R of soap which result from the operation of the cutting device 70 and/or the pressing molds or dies may optionally be recycled back to the plodder 50 or any other portion of the system.

The soap product P (also called soap herein) may be used for treatment of seborrheic keratoses, keratosis pilaris, keratoderma, and/or related conditions by rubbing the soap over the seborrheic keratoses, keratosis pilaris, keratoderma, and/or related conditions. Water may be added to the soap or the area to produce suds and expose the abrasive particles in the soap. The soap bar may be manually brushed in a to and fro fashion across the rough skin. The area may be washed off with water after use. The amount of pressure applied may be proportional to the thickness of the rough skin of the seborrheic keratoses, keratosis pilaris, keratoderma and/or related conditions prior to or during the rubbing. Water may be added to the soap or the area with the seborrheic keratoses, keratosis pilaris, keratoderma, and/or related conditions prior to or during the rubbing.

In some embodiments, the soap disclosed herein may be optionally included with (and somehow attached to) and used in combination with a rope as “soap on a rope,” similar to the soap on a rope by Aramis. In other embodiments, the soap disclosed herein may optionally be attached to a stick to allow a user to effectively reach hard-to-reach areas on the skin with the soap using the stick. Either the stick or rope may be used to help the user reach hard-to reach places such as the user's back.

While the foregoing is directed to embodiments of the invention, other and further embodiments of the invention may be devised without departing from the basic scope thereof, and the scope thereof is determined by the claims that follow.

1. Soap for treating seborrheic keratoses, keratosis pilaris, keratoderma, or related conditions, comprising:
a soap base comprising one or more oils or fatty acids; and
additives comprising:
pumice, wherein a weight percent of pumice in the soap is in a range of from approximately two percent to approximately three percent, and
polyethylene, wherein a weight percent of polyethylene in the soap is in a range of from approximately one percent to approximately two percent.

2. The soap of claim 1, wherein a weight percent of pumice in the soap is approximately two percent and a weight percent of polyethylene in the soap is approximately two percent.

3. The soap of claim 1, wherein the soap base further comprises one or more preservatives or one or more chelating agents.

4. The soap of claim 3, wherein the one or more preservatives or one or more chelating agents comprise tetrasodium ethylenediamine tetraacetic acid or tetrasodium etidronate.

5. The soap of claim 1, wherein the one or more oils are one or more vegetable oils.

6. The soap of claim 1, wherein the one or more oils comprise sodium palmate or sodium cocoate.

7. The soap of claim 1, wherein the additives further comprise palmitic acid.

8. The soap of claim 1, wherein the additives further comprise coconut acid.

9. The soap of claim 1, wherein a weight percent of pumice in the soap is approximately three percent and a weight percent of polyethylene in the soap is approximately two percent.

10. The soap of claim 1, wherein a ratio of the weight percent of pumice in the soap to the weight percent of polyethylene in the soap is 2:1.

11. The soap of claim 10, wherein the weight percent of pumice in the soap is approximately two percent of the total weight of the soap.

12. The soap of claim 11, wherein the weight percent of polyethylene in the soap is approximately one percent of the total weight of the soap.

13. (canceled)

14. (canceled)

15. (canceled)

16. (canceled)

17. (canceled)

18. (canceled)

19. (canceled)

20. (canceled)

21. Soap for treating seborrheic keratoses, keratosis pilaris, keratoderma, or related condition, comprising:
a soap base comprising one or more oils or fatty acids; and
additives comprising pumice and polyethylene in effective amounts for effectively treating the seborrheic keratoses, the keratosis pilaris, the keratoderma, or the related condition upon rubbing of the soap on the seborrheic keratoses, the keratosis pilaris, the keratoderma, or the related condition.

22. The soap of claim 21, wherein the effective amount of pumice is a weight percent of pumice in a range of from approximately two percent to approximately three percent of the soap.

23. The soap of claim 22, wherein the effective amount of polyethylene is a weight percent of polyethylene in a range of from approximately one percent to approximately two percent of the soap.

24. The soap of claim 21, wherein the effective amounts are a 2:1 ratio of the weight percent of pumice in the soap to the weight percent of polyethylene in the soap.

25. The soap of claim 1, further comprising:
palmitic acid or coconut acid;
water; and
one or more chelating agents.

26. The soap of claim 25, wherein a weight percent of the water in the soap is approximately twelve percent.

27. The soap of claim 26, wherein a weight percent of the palmitic acid in the soap is approximately 4 percent.

28. The soap of claim 26, wherein a weight percent of the one or more chelating agents in the soap is less than 0.05 percent.
